(19) United States
(12) Patent Application Publication
(10) Pub. No.: US 2017/0360864 A1
(43) Pub. Date: Dec. 21, 2017

(54) METHOD FOR INCREASING EXPRESSIONS OF CLOCK GENE, ARNTL GENE, AND/OR PER2 GENE BY USING MOMORDICA CHARANTIA EXTRACT

(71) Applicant: TCI CO., LTD, Taipei (TW)

(72) Inventors: Yung-Hsiang LIN, Taipei (TW); I-Hui CHEN, Taipei (TW)

(21) Appl. No.: 15/629,058

(22) Filed: Jun. 21, 2017

Related U.S. Application Data
(60) Provisional application No. 62/352,767, filed on Jun. 21, 2016.

(51) Int. Cl. A61K 36/42

(52) U.S. Cl. CPC A61K 36/42

(57) ABSTRACT

A method for increasing the expressions of CLOCK, ARNTL and/or PER2 genes is provided, wherein the method comprises administering to a subject in need an effective amount of Momordica charantia extract. The method is useful for treating or preventing diseases related to CLOCK, ARNTL and/or PER2 genes, or regulating physiological functions related to CLOCK, ARNTL and/or PER2 genes. The method is especially for adjusting the biological clock, improving sleep quality, and facilitating sleep.

### FIELD OF THE INVENTION

The present invention relates to the use of a Momordica charantia extract, especially a roasted Momordica charantia extract, the use includes treating or preventing diseases related to CLOCK gene, ARNTL gene, and/or PER2 gene, or regulating physiological functions related to the genes. The present invention especially relates to the use of the extract in at least one of adjusting the biological clock, improving sleep quality, and facilitating sleep.

### BACKGROUND OF THE INVENTION

Circadian rhythm is a change of a roughly 24 hour cycle in vital activities (e.g., food intake, body activities, sleep, awakening of animals), which is driven by a circadian clock. When the circadian rhythm of human bodies is disturbed, it will lead to changes in the body's biological clock, decrease in sleep quality, irregularity of hormonal secretion, and decrease in the ability to act. Thus, work efficiency will be reduced, probability of accidents will increase, and many diseases (e.g., depressive disorder) may be induced.

Currently, Diazepam and Lorazepam are common drugs used in clinic for regulating circadian rhythm to treat related diseases such as insomnia and depressive disorder. However, patients are prone to addiction after using the aforementioned drugs and may have side effects, such as hypersomnia, nausea, headache, vomiting, gastrointestinal discomfort, memory impairment, rebound insomnia, unconsciousness, ataxia, dyspnea, and/or somnambulism. Therefore, there is necessity and urgency for continuously developing a drug or method for regulating circadian rhythm effectively without causing addictions and side effects.

Researchers have found that a human body's circadian rhythm and sleep cycle are regulated by the expressions of genes including CLOCK (circadian locomotor output cycles kaput), ARNTL (aryl hydrocarbon receptor nuclear translocator like), and PER2 (period circadian clock 2). Thus, if the expressions of CLOCK gene, ARNTL gene, and PER2 gene could be increased, the circadian rhythm will be effectively regulated which is beneficial to adjust the biological clock, improve sleep quality and facilitate sleep.

Inventors of the present invention found that Momordica charantia extract is effective in regulating the expressions of CLOCK, ARNTL, and PER2 genes, and thus, can be used for adjusting the biological clock, improving sleep quality, and facilitating sleep, as well as be used for treating or preventing diseases related to the aforementioned genes, or regulating physiological functions related to the aforementioned genes. Preferably, the Momordica charantia extract is an extract of roasted Momordica charantia.

### SUMMARY OF THE INVENTION

An objective of the present invention is to provide a use of Momordica charantia extract in the manufacture of a medicament, wherein the medicament is for increasing the expressions of at least one of CLOCK, ARNTL, and PER2 genes. Preferably, the medicament is used for treating or preventing diseases related to the aforementioned genes, or regulating physiological functions related to the aforementioned genes. The disease related to CLOCK gene, ARNTL gene, and/or PER2 gene is at least one of cancer (including gastric cancer, lung cancer, and ovarian cancer), alcohol-induced liver injury, depressive disorder, bipolar disorder, male infertility, aging, Parkinson's disease, vascular disease, metabolic syndrome (including obesity, hypertension, and diabetes mellitus), and cocaine addiction. The physiological function related to CLOCK gene, ARNTL gene, and/or PER2 gene is at least one of glycogen synthesis, lung pathophysiology, maintenance and adaption of skeletal muscles, maintenance and differentiation of pluripotent stem cells, glucose homeostasis, glucose metabolism, insulin release, adipogenesis, retinal function, steroidogenesis, hepatic lipid metabolism, angiogenesis, autophagy in aging, maintenance of bone volume and bone density, circadian clock, diurnal preference, glycogen metabolism, lipid metabolism, endothelial progenitor cell function, and oxidative injury. Preferably, the Momordica charantia extract is an extract of roasted Momordica charantia.

Another objective of the present invention is to provide a use of Momordica charantia extract in the manufacture of a food product, wherein the food product can be used for adjusting the biological clock, improving sleep quality, and facilitating sleep. Preferably, the Momordica charantia extract is an extract of roasted Momordica charantia.

Still another objective of the present invention is to provide a method for increasing the expressions of CLOCK gene, ARNTL gene, and/or PER2 gene in a subject in need, comprising administering to the subject an effective amount of a Momordica charantia extract. The method is for treating or preventing diseases related to the aforementioned genes, or regulating physiological functions related to the aforementioned genes. The method is for adjusting the biological clock, improving sleep quality, and facilitating sleep. Preferably, the Momordica charantia extract is an extract of roasted Momordica charantia.

Yet another objective of the present invention is to provide a method for providing a Momordica charantia extract with enhanced effect on increasing the expressions of CLOCK gene, ARNTL gene, and/or PER2 gene, comprising subjecting Momordica charantia to a roasting treatment prior to extracting the Momordica charantia. Preferably, the roasting treatment comprises roasting at a stepwise-increased temperature.

The detailed technology and some particular embodiments implemented for the present invention are described in the following paragraphs for people skilled in this field to well appreciate the features of the claimed invention.

### BRIEF DESCRIPTION OF THE DRAWINGS

FIG. 1 shows the expression levels of CLOCK, ARNTL, and PER2 genes in the HepG2 cells of different groups (as compared to the expression level of the control group), wherein the cells of “control group” were cultivated in a medium free of any Momordica charantia extract for 48 hours, the cells of “6 hours group” and “24 hours group” were cultivated in a medium free of any Momordica charantia extract for 24 hours, and then an unroasted Momordica charantia extract was added into the medium to continue the cultivation for another 6 hours and 24 hours respectively, and the cells of “48 hours group” were cultivated in a medium containing unroasted Momordica charantia extract for 48 hours.

FIG. 2 shows the relative expression level of CLOCK gene in HepG2 cells of different groups (as compared to the expression level of the control group), wherein the cells of the “unroasted groups” were cultivated in the presence of an unroasted Momordica charantia extract while the cells of the “roasted 1 groups” to “roasted 5 groups” were cultivated in the presence of a roasted Momordica charantia extract.

FIG. 3 shows the expression level of ARNTL gene of HepG2 cells in the “unroasted groups” and the “roasted 1 groups” to “roasted 5 groups” described above (as compared to the expression level of the control group).

FIG. 4 shows the expression level of PER2 gene of HepG2 cells in the “unroasted groups” and the “roasted 1 groups” to “roasted 5 groups” described above (as compared to the expression level of the control group).

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification or defined in the appended claims.

In addition, unless otherwise indicated herein, the expressions “a,” “an,” “the,” or the like recited in the specification of the present invention (especially in the claims) are intended to include both the singular and plural forms. The term “treat” or “treating” recited in this specification should not be construed as treating a subject until the subject is completely recovered, but should include maintaining the progression or symptoms of the diseases in a substantially static state, increasing the recovery rate of a subject, alleviating the severity of a particular condition of illness, or increasing the quality of life of a patient. The term “prevent” or “preventing” recited in this specification refers to inhibiting or preventing a particular condition of illness from breaking out, or maintaining good health in a sensitive subject to tolerate diseases. The term “regulate” or “regulating” recited in this specification refers to upregulating (includes inducing, stimulating, and enhancing) or downregulating (includes inhibiting and weakening) the physiological functions in a subject toward a normal state. The term “an effective amount” recited in this specification refers to the amount of the substance that can at least partially alleviate the condition that is being treated in a suspected subject when administered to the subject. The term “subject” recited in this specification refers to a mammalian, including human and non-human animals.

Momordica charantia is a plant belonging to the family Cucurbitaceae. In general, Momordica charantia originally habitats in the tropics, and is widely planted in South Asia, Southeast Asia, Taiwan, China, and Caribbean islands. In Taiwan, Momordica charantia is commonly distributed in Taichung, Changhua, Chiayi, and Kaohsiung.

As described above, inventors of the present invention found that the Momordica charantia extract is effective in regulating the expressions of CLOCK, ARNTL, and PER2 genes. It has been known that CLOCK gene is positively related to the treatment, prevention, or regulation of alcohol-induced liver injury, cancer, depressive disorder, glycogen synthesis, lung pathophysiology, male infertility, obesity, as well as maintenance and adaption of skeletal muscles, maintenance and differentiation of pluripotent stem cells. Therefore, if the expression of CLOCK gene can be increased, the diseases related to CLOCK gene can be treated or prevented, and the physiological functions related to CLOCK gene can be regulated. The correlation between CLOCK gene and the aforementioned diseases and physiological functions can be seen in, for example, “The Molecular Circadian Clock and Alcohol-Induced Liver Injury. Biomolecules. 5: 2504-2537 (2015);” “Genetic variation of clock genes and cancer risk: a field synopsis and meta-analysis. Oncotarget. Vol. 8, (No. 14), pp: 23978-23995 (2017);” “CLOCK is suggested to associate with comorbid alcohol use and depressive disorders. Journal of Circadian Rhythms. 8:1 (2010);” “CLOCK Regulates Circadian Rhythms of Hepatic Glycogen Synthesis through Transcriptional Activation of Gys2. The journal of biological chemistry. Vol. 285, No. 29, pp. 22114-22121 (2010);” “Circadian molecular clock in lung pathophysiology. Am J Physiol Lung Cell Mol Physiol. 309: L1056-L1075 (2015);” “Genetic Variation in Circadian Rhythm Genes CLOCK and ARNTL as Risk Factor for Male Infertility. PLoS One. 8(3):e59220 (2013);” “Altered Clock Gene Expression in Obese Visceral Adipose Tissue Is Associated with Metabolic Syndrome. PLoS One. November 3; 9(11):e111678 (2014);” “Circadian Rhythms, the Molecular Clock, and Skeletal Muscle. Curr Top Dev Biol. 96: 231-271 (2011);” and “Role of circadian gene Clock during differentiation of mouse pluripotent stem cells. Protein Cell. 7(10:820-832 (2016),” which are entirely incorporated hereinto by reference.

It has been known that ARNTL gene is positively related to the treatment, prevention, or regulation of obesity, adipogenesis, aging, bipolar disorder, cancer, glucose homeostasis, glucose metabolism, hypertension, insulin release, male infertility, Parkinson's disease, retinal function, steroidogenesis, type II diabetes mellitus, and vascular disease. Therefore, if the expression of ARNTL gene can be increased, the diseases related to ARNTL gene can be treated or prevented, and the physiological functions related to ARNTL gene can be regulated. The correlation between ARNTL gene and the aforementioned diseases or physiological functions can be seen in, for example, “Obesity in mice with adipocyte-specific deletion of clock component Arntl. Nat Med. 18(12): 1768-1777 (2012);” “Brain and muscle Arnt-like protein-1 (BMAL1), a component of the molecular clock, regulates adipogenesis. Proc Natl Acad Sci USA. 102(34):12071-6 (2005);” “Early aging and age-related pathologies in mice deficient in BMAL1, the core component of the circadian clock. Genes Dev. 20(14):1868-73 (2006);” “Suggestive evidence for association of the circadian genes PERIOD3 and ARNTL with bipolar disorder. Am J Med Genet B Neuropsychiatr Genet. 141B(3): 234-241 (2006);” “A Large Scale shRNA Barcode Screen Identifies the Circadian Clock Component ARNTL as Putative Regulator of the p53 Tumor Suppressor Pathway. PLoS One. 4(3):e4798 (2009);” “BMAL1 and CLOCK, Two Essential Components of the Circadian Clock, Are Involved in Glucose Homeostasis. PLoS Biol. November 2(11):e377 (2004);” “Global Loss of Bmal1 Expression Alters Adipose Tissue Hormones, Gene Expression and Glucose Metabolism. PLoS One. June 4; 8(6):e65255 (2013);” “Aryl hydrocarbon receptor nuclear translocator-like (BMAL1) is associated with susceptibility to hypertension and type 2 diabetes. Proc Natl Acad Sci USA. 104(36):14412-7 (2007);” “Aryl Hydrocarbon Receptor Nuclear Translocator/Hypoxiainducible Factor-1β Plays a Critical Role in Maintaining Glucose-stimulated Anaplerosis and Insulin Release from Pancreatic β-Cells. J Biol Chem. 286(2):1014-24 (2011);” “Genetic variation in circadian rhythm genes CLOCK and ARNTL as risk factor for male infertility. PLoS One. 8(3):e59220 (2013);” “Association of ARNTL and PER1 genes with Parkinson's disease: a case-control study of Han Chinese. Sci Rep. 5:15891 (2015);” “Intrinsic circadian clock of the mammalian retina: importance for retinal processing of visual information. Cell. 130(4):730-41 (2007);” “Impaired steroidogenesis and implantation failure in Bmal1−/− mice. Endocrinology. April; 150(4):1879-85 (2009);” “Aryl hydrocarbon receptor nuclear translocator-like (BMAL1) is associated with susceptibility to hypertension and type 2 diabetes. Proc Natl Acad Sci USA. September 4; 104(36):14412-7 (2007);” and “Vascular disease in mice with a dysfunctional circadian clock. Circulation. 119(11):1510-7 (2009),” which are entirely incorporated hereinto by reference.

It has been known that PER2 gene is positively related to the treatment, prevention, or regulation of hepatic lipid metabolism, metabolic syndrome, angiogenesis, autophagy in aging, bone volume and bone density, aging, circadian clock, cocaine addiction, diabetes mellitus, diurnal preference, endothelial progenitor cell function, gastric cancer, glycogen metabolism, lung cancer, ovarian cancer, and oxidative injury. The correlation between PER2 gene and the aforementioned diseases and physiological functions can be seen in, for example, “KSRP is critical in governing hepatic lipid metabolism through controlling Per2 expression. J Lipid Res. 56(2):227-40. (2015);” “NPAS2 and PER2 are linked to risk factors of the metabolic syndrome. J Circadian Rhythms. 26; 7:5. (2009);” “Period 2 is essential to maintain early endothelial progenitor cell function in vitro and angiogenesis after myocardial infarction in mice. J Cell Mol Med. 18(5):907-18 (2014);” “Crosstalk of clock gene expression and autophagy in aging. Aging (Albany N.Y.). 8(9):1876-1895 (2016);” “Decreased Bone Volume and Bone Mineral Density in the Tibial Trabecular Bone Is Associated with Per2 Gene by 405 nm Laser Stimulation. Int J Mol Sci. 16(11):27401-10 (2015);” “Negative reciprocal regulation between Sirt1 and Per2 modulates the circadian clock and aging. Sci Rep. 6:28633. (2016);” “Repeat variation in the human PER2 gene as a new genetic marker associated with cocaine addiction and brain dopamine D2 receptor availability. Transl Psychiatry. 2:e86. (2012);” “Per2 mutation recapitulates the vascular phenotype of diabetes in the retina and bone marrow. Diabetes. 62(1):273-82. (2013);” “PER2 Variation is Associated with Diurnal Preference in a Korean Young Population. Behav Genet. 41(2):273-7. (2011);” “Period 2 is essential to maintain early endothelial progenitor cell function in vitro and angiogenesis after myocardial infarction in mice. J Cell Mol Med. 18(5):907-18. (2014);” “Prognostic relevance of Period1 (Per1) and Period2 (Per2) expression in human gastric cancer. Int J Clin Exp Pathol. 15; 7(2):619-30. (2014);” “PER2 promotes glucose storage to liver glycogen during feeding and acute fasting by inducing Gys2 PTG and G L expression. Mol Metab. 2(3):292-305. (2013);” “PER2 controls lipid metabolism by direct regulation of PPARγ. Cell Metab. 12(5):509-20. (2010);” “Aberrant expression of Per1, Per2 and Per3 and their prognostic relevance in non-small cell lung cancer. Int J Clin Exp Pathol. 7(11):7863-71 (2014);” “Effects of Per2 overexpression on growth inhibition and metastasis, and on MTA1, nm23-H1 and the autophagy-associated PI3K/PKB signaling pathway in nude mice xenograft models of ovarian cancer. Mol Med Rep. 13(6):4561-8. (2016);” “The Mammalian circadian clock gene pert modulates cell death in response to oxidative stress. Front Neurol. 5:289. (2015);” and “Loss of corepressor PER2 under hypoxia up-regulates OCT1-mediated EMT gene expression and enhances tumor malignancy. Proc Natl Acad Sci USA. 110(30):12331-6 (2013),” which are entirely incorporated hereinto by reference.

Thus, the present invention provides a use of Momordica charantia extract in the manufacture of a medicament, wherein the medicament is used for increasing the expressions of at least one of CLOCK, ARNTL, and PER2 genes. Preferably, the medicament is used for treating or preventing diseases related to the aforementioned genes, or regulating physiological functions related to the aforementioned genes.

The disease related to CLOCK gene, ARNTL gene, and/or PER2 gene is at least one of cancer (including gastric cancer, lung cancer, and ovarian cancer), alcohol-induced liver injury, depressive disorder, bipolar disorder, male infertility, aging, Parkinson's disease, vascular disease, metabolic syndrome (including obesity, hypertension, and diabetes mellitus), and cocaine addiction.

The physiological function related to CLOCK gene, ARNTL gene, and/or PER2 gene is at least one of glycogen synthesis, lung pathophysiology, maintenance and adaption of skeletal muscles, maintenance and differentiation of pluripotent stem cells, glucose homeostasis, glucose metabolism, insulin release, adipogenesis, retinal function, steroidogenesis, hepatic lipid metabolism, angiogenesis, autophagy in aging, maintenance of bone volume and bone density, circadian clock, diurnal preference, glycogen metabolism, lipid metabolism, endothelial progenitor cell function, and oxidative injury.

Furthermore, inventors of the present invention found that in the uses according to the present invention, if an extract of roasted Momordica charantia (i.e., Momordica charantia is subjected to a roasting treatment prior to being extracted) is used as the Momordica charantia extract, the desired effects on increasing the expressions of CLOCK gene, ARNTL gene, and/or PER2 gene can be further enhanced. Thus, an extract of roasted Momordica charantia is preferred to be adopted in the above uses.

Therefore, the present invention also provides a method for providing a Momordica charantia extract with enhanced effects. The method comprises subjecting Momordica charantia to a roasting treatment prior to extracting the Momordica charantia. Preferably, the roasting treatment is conducted at a high temperature. For example, the roasting treatment is conducted at a temperature ranging from 80 to 200° C. More preferably, the roasting treatment is conducted at a stepwise-increased temperature. For example, the roasting treatment comprises at least two stages, i.e., a first roasting stage at T1 and a second roasting stage at T2 after the first roasting stage, wherein T1<T2. Preferably, T1<T2, 80° C.≦T1<200° C., and 80° C.<T2≦200° C.; and more preferably, T1<T2, 80° C.≦T1≦135° C., and 135° C.≦T2≦200° C.

In the method for providing a Momordica charantia extract with enhanced effects according to the present invention, the roasting time could be adjusted optionally, as long as the water content of Momordica charantia could be reduced. In some embodiments of the present invention, a roasting treatment comprising two stages with a stepwise-increased temperature was adopted, wherein the Momordica charantia was subjected to a first roasting stage at an environment ranging from 90 to 120° C. for 2 to 24 hours, and then to a second roasting stage at an environment ranging from 150 to 180° C. for 20 to 100 minutes.

In the method and use according to the present invention, the Momordica charantia could be extracted by any suitable method to provide a Momordica charantia extract needed by the present invention. For example, the Momordica charantia could be directly extracted with water, followed by a solid-liquid separating approach (e.g., filtration) to obtain a liquid extract. Optionally, the liquid extract could be further subjected to the other operations such as concentration and/or drying to obtain a Momordica charantia extract in a form of liquid or solid.

Depending on the desired purpose, the medicament provided according to the present invention could be provided in any suitable form without specific limitations. For example, the medicament could be administered to a subject in need by an oral or parenteral (such as transdermal administration, nasal administration, subcutaneous injection, intravenous injection, muscular injection, peritoneal injection, subcutaneous implantation, or interstitial implantation) route, but the administration is not limited thereby. Depending on the form and purpose, suitable carriers could be chosen and used to provide the medicament, as long as the carriers do not adversely affect the desired effects of the Momordica charantia extract of the present invention. For example, the carriers could be, but are not limited to excipients, diluents, auxiliaries, stabilizers, absorbent retarders, disintegrating agents, hydrotropic agents, emulsifiers, antioxidants, adhesives, binders, tackifiers, dispersants, suspending agents, lubricants, hygroscopic agents, etc.

As a dosage form suitable for oral administration, examples of the carriers include, but are not limited to, water, saline, dextrose, glycerol, ethanol or its analogs, cellulose, starch, sugar bentonite, and combinations thereof. The medicament could be provided in any suitable form for oral administration, such as in a solid form of a tablet, a pill, a capsule, granules, a pulvis, etc., or in a liquid form of an oral liquid, a syrup, a spirit, an elixir, a tincture, etc., but the form is not limited thereby.

As for the form of injection or drip suitable for subcutaneous, intravenous, muscular, or peritoneal administration, the medicament provided according to the present invention could comprise one or more ingredient(s), such as an isotonic solution, a salt-buffered saline (e.g., phosphate-buffered saline or citrate-buffered saline), a hydrotropic agent, an emulsifier, 5% sugar solution, and other carriers to provide the medicament as an intravenous infusion, an emulsified intravenous infusion, a powder for injection, a suspension for injection, or a powder suspension for injection, etc. Alternatively, the medicament could be prepared as a pre-injection solid. The pre-injection solid could be provided in a form which is soluble in other solutions or suspensions, or in an emulsifiable form. A desired injection is provided by dissolving the pre-injection solid in other solutions or suspensions or emulsifying it prior to being administered to a subject in need. In addition, as for the external dosage form for nasal or transdermal administration, the medicament could be provided in the form of, for example, a liniment (such as an emulsion, a cream, a gel, a dispersing paste, an ointment), a spray, a patch, or a solution (such as a cleaning liquid, a suspension).

In the medicament provided according to the use of the present invention, the ratio of the amount of Momordica charantia extract in the medicament could be adjusted depending on practical requirements.

Optionally, the medicament provided according to the present invention could further comprise a suitable amount of additives, such as flavoring agent, a toner, or a coloring agent for enhancing the palatability and the visual perception of the medicament, and a buffer, a conservative, a preservative, an antibacterial agent, or an antifungal agent for improving the stability and storability of the medicament. In addition, the medicament could optionally further comprise one or more other active ingredient(s), or be used in combination with a medicament comprising one or more other active ingredient(s), to further enhance the effect of the medicament, or to increase the application flexibility and adaptability of the preparation thus provided, as long as the other active ingredients do not adversely affect the desired effects of Momordica charantia extract.

Depending on the need, age, body weight, and health conditions of the subject, the medicament provided according to the present invention could be dosed with various administration frequencies, such as once a day, multiple times a day, or once every few days, etc.

As described above, inventors of the present invention found that the Momordica charantia extract is effective in regulating the expressions of CLOCK, ARNTL, and PER2 genes, and thus, can be used for adjusting the physiological clock, improving sleep quality, and facilitating sleep. Therefore, the present invention also provides a use of Momordica charantia extract in the manufacture of a food product, wherein the food product is used for at least one of adjusting the physiological clock, improving sleep quality, and facilitating sleep.

The food product provided according to the present invention could be a health food, a nutritional supplement food, or a special nutritional food. The food product may be provided as dairy products, meat products, breadstuff, pasta, cookies, troche, capsule, fruit juices, teas, sport beverages, nutritional beverages, etc., but is not limited thereby. Preferably, the food product according to the present invention is provided as a health food.

Depending on the age, body weight and health conditions of the subject, the health food, nutritional supplement food and special nutritional food provided according to the present invention could be taken in various frequencies, such as once a day, multiple times a day or once every few days, etc. The amount of Momordica charantia extract in the health food, nutritional supplement food and special nutritional food provided according to the present invention could be adjusted, preferably to the amount that should be taken daily, depending on the specific population.

The recommended daily dosage, use standards and use conditions for a specific population (e.g., insomnia, circadian desynchronization), or the recommendations for a use in combination with another food product or medicament could be indicated on the exterior package of the health food, nutritional supplement food and/or special nutritional food provided by the present invention. Thus, it is suitable for the user to take the health food, nutritional supplement food and/or special nutritional food by him- or herself safely and securely without the instruction of a doctor, pharmacist, or related executive. In the food product provided according to the present invention, the type, suitable dosage and uses in related treatment of Momordica charantia extract are all in line with the above description.

The present invention also provides a method for increasing the expressions of CLOCK gene, ARNTL gene, and/or PER2 gene in a subject in need, comprising administering to the subject an effective amount of Momordica charantia extract. The type, applied route, applied form, applied frequency and uses in related treatment of Momordica charantia extract are all in line with the above description.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Preparation Examples

A. Preparation of Unroasted Momordica charantia Extract

Momordica charantia was washed, and then soaked in water (Momordica charantia: water=1:5 in weight) and stewed at 80° C. for 30 minutes to provide a crude liquid extract. Then, the crude liquid extract was filtrated with a 200 mesh filter to provide a liquid extract. Finally, the liquid extract was concentrated four folds to provide an extract (hereinafter referred to as “unroasted Momordica charantia extract”).

B. Preparation of Roasted Momordica charantia Extract

Momordica charantia was washed and divided into five groups. Those five groups were respectively roasted with the following conditions of Table 1 (instrument: OV-80 oven, purchased from Firstek company):

TABLE 1

| Groups | Roasting conditions |
|---|---|
| Group 1 | Roasted at 120° C. for 2 hours, and then roasted at 150° C. |
| | for 80 minutes |
| Group 2 | Roasted at 120° C. for 2 hours, and then roasted at 150° C. |
| | for 100 minutes |
| Group 3 | Roasted at 120° C. for 2 hours, and then roasted at 180° C. |
| | for 40 minutes |
| Group 4 | Roasted at 120° C. for 2 hours, and then roasted at 150° C. |
| | for 60 minutes |
| Group 5 | Roasted at 90° C. for 1 day, and then roasted at 180° C. |
| | for 20 minutes |

Thereafter, the roasted Momordica charantia of above each group was subjected to the operating procedures (including extraction, filtration, and concentration) as [Preparation Example A] to provide an extract (hereinafter referred to as “roasted Momordica charantia extract”).

C. Treatment of Cells

HepG2 cells (provided by American Type Culture Collection (ATCC); number: ATCC® HB-8065) were cultivated in a Dulbecco's modified Eagle's medium (DMEM; purchased from Gibco company) containing 10% bovine serum albumin (purchased from Gibco company) for 24 hours. The cells thus provided were used in the following experiments.

### Example 1

### Effects of Momordica charantia Extract on the Expression Levels of CLOCK, ARNTL, and PER2 Genes

(1-1) Effects of Unroasted Momordica charantia Extract

HepG2 cells obtained from [Preparation Example C] were divided into four groups and were treated as follows under 5% CO₂ at 37° C.:
(A) “Control group”: cells were cultivated in a DMEM medium containing 10% bovine serum albumin (i.e., a medium free of any Momordica charantia extract) for 48 hours.
(B) “6 hours group”: cells were cultivated in a medium free of any Momordica charantia extract for 24 hours, and then the unroasted Momordica charantia extract provided by [Preparation Example A] was added into the medium to provide a final concentration of 2 mg/ml and continue the cultivation for another 6 hours.
(C) “24 hours group”: cells were cultivated in a medium free of any Momordica charantia extract for 24 hours, and then the unroasted Momordica charantia extract provided by [Preparation Example A] was added into the medium to provide a final concentration of 2 mg/ml and continue the cultivation for another 24 hours.
(D) “48 hours group”: cells were cultivated in a DMEM medium containing 10% bovine serum albumin for 48 hours, but the unroasted Momordica charantia extract provided by [Preparation Example A] was added into the medium to provide a final concentration of 2 mg/ml before conducting the cultivation.

Thereafter, cells of the above groups were harvested and subjected to RNA extraction with an RNA Lysis buffer (purchased from Geneaid company). The RNA was then transcribed into cDNA with a SuperScript™ Reverse Transcriptase kit (purchased from Invitrogen company). Thereafter, the aforementioned cDNA was subjected to a real-time quantitative polymerase chain reaction (Q-PCR) by a ABI StepOnePlus™ system (purchased from Applied Biosystems) to determine the expression levels of CLOCK, PER2, ARNTL, and RPLP0 genes in the cells of each group. Then, the expression level of each gene was normalized by using the expression level of RPLP0 gene as a basis. Lastly, the gene expression level of each group was normalized by using that of the control group as a basis. The results are shown in FIG. 1.

As shown in FIG. 1, as compared to the control group (whose gene expression level was served as 1-fold), the expression level of CLOCK gene in the cells of the 48 hours group is 1.35-fold that of the control group, the expression level of ARNTL gene in the cells of the 48 hours group is 4.36-fold that of the control group, and the expression level of PER2 gene in the cells of the 48hours group is 2.83-fold that of the control group. These results indicate that the Momordica charantia extract can significantly increase the expressions of CLOCK, ARNTL, PER2 genes of hepatic cells, and thus, can be used for regulating the human body's physiological clock and circadian rhythm.

(1-2) Effects of Roasted Momordica charantia Extract

HepG2 cells obtained from [Preparation Example C] were divided into six groups and were treated as follows under 5% CO₂ at 37° C.:
(A) “Unroasted group”: cells were further divided into three subgroups and separately cultivated in a DMEM medium containing 10% bovine serum albumin for 24 hours, and then the unroasted Momordica charantia extract provided by [Preparation Example A] was added into the medium to provide a final concentration of 2 mg/ml and continue the cultivations of the three subgroups for another 6 hours, 24 hours, and 48 hours, respectively.
(B) “Roasted 1 group”: cells were treated as the “unroasted group”, but the unroasted Momordica charantia extract provided by [Preparation Example A] was replaced with roasted Momordica charantia extract of Group 1 provided by [Preparation Example B].
(C) “Roasted 2 group”: cells were treated as the “unroasted group”, but the unroasted Momordica charantia extract provided by [Preparation Example A] was replaced with roasted Momordica charantia extract of Group 2 provided by [Preparation Example B].
(D) “Roasted 3 group”: cells were treated as the “unroasted group”, but the unroasted Momordica charantia extract provided by [Preparation Example A] was replaced with roasted Momordica charantia extract of the Group 3 provided by [Preparation Example B].
(E) “Roasted 4 group”: cells were treated as the “unroasted group”, but the unroasted Momordica charantia extract provided by [Preparation Example A] was replaced with roasted Momordica charantia extract of Group 4 provided by [Preparation Example B].
(F) “Roasted 5 group”: cells were treated as the “unroasted group”, but the unroasted Momordica charantia extract provided by [Preparation Example A] was replaced with roasted Momordica charantia extract of Group 5 provided by [Preparation Example B].

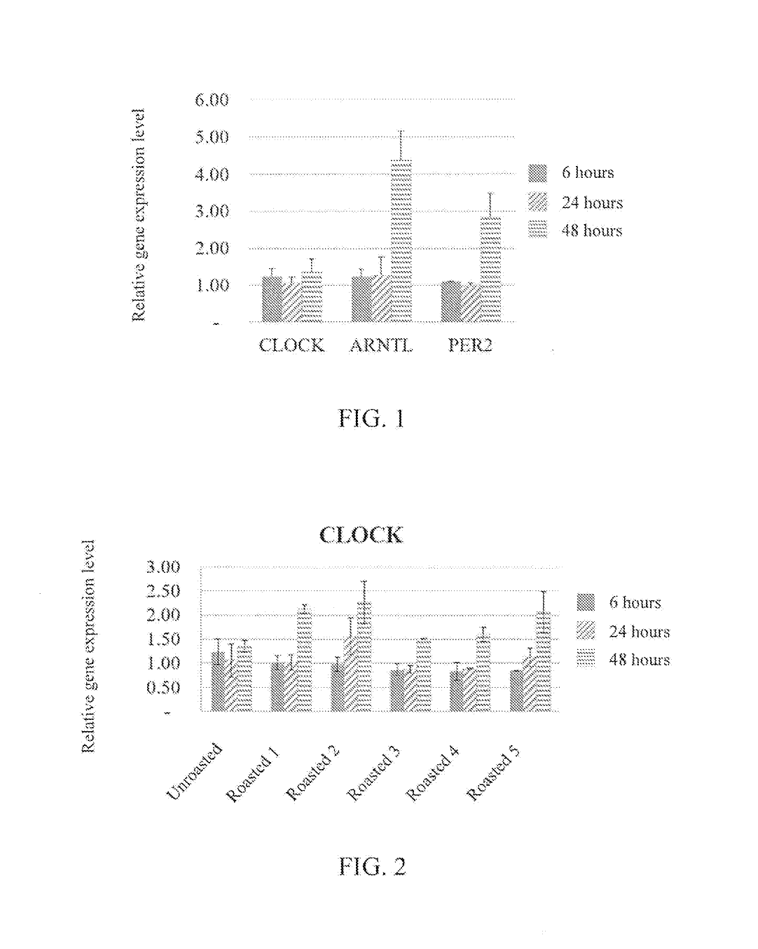

Cells of the above groups were harvested and subjected to RNA extraction, cDNA preparation, and Q-PCR in the order as described in (1-1). Then, the expression levels of CLOCK, PER2, ARNTL genes in the cells of each group were determined. The results are shown in FIGS. 2 to 4, which also had been normalized by using the gene expression level of the control group (i.e., cells without being treated with Momordica charantia extract) as a basis.

As shown in FIGS. 2 to 4, both the unroasted and roasted Momordica charantia extract can exert the best effects of regulating CLOCK, ARNTL, PER2 genes on hepatic cells by a 48 hour-treatment.

Besides, the changing trends of the expression levels of CLOCK, ARNTL, PER2 genes in Momordica charantia would vary along with the roasting condition of Momordica charantia. As shown in FIG. 2, as compared to the unroasted group, the expression level of CLOCK gene in the cells of the roasted 1 groups to roasted 5 groups all significantly increased. For example, according to the results of the 48 hour-treatment, the expression level of CLOCK gene in the cells of the unroasted group is 1.35-fold that of the control group, and the expression levels of CLOCK gene in the cells of the roasted 1 group to roasted 5 group are respectively 2.12-, 2.26-, 1.50-, 1.60-, and 2.07-fold that of the control group.

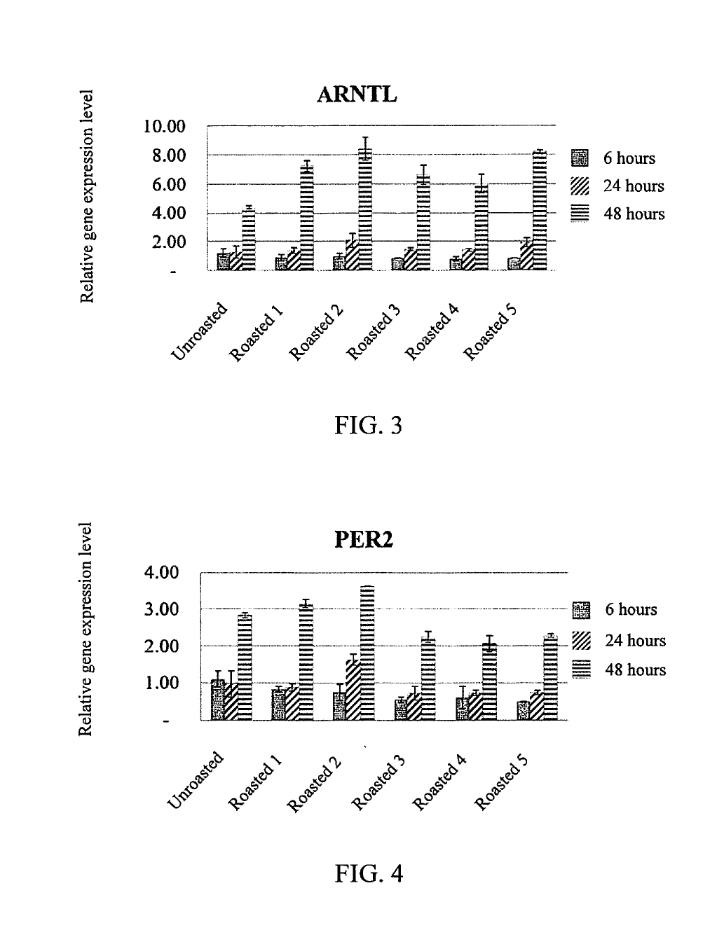

As shown in FIG. 3, according to the results of the 48 hour-treatment, the expression level of ARNTL gene in the cells of the unroasted group is 4.36-fold that of the control group, and the expression levels of ARNTL gene in the cells of the roasted 1 group to roasted 5 group are respectively 7.24-, 8.41-, 6.61-, 6.01-, and 8.19-fold that of the control group.

As shown in FIG. 4, according to the results of the 48 hours-treatment, the expression level of PER2 gene in the cells of the unroasted group is 2.83-fold that of the control group. The expression levels of PER2 gene in the cells of the roasted 1 group to roasted 5 group are respectively 3.14-, 3.62-, 2.24-, 2.06-, and 2.27-fold that of the control group.

The above results indicate that, as compared to the unroasted Momordica charantia extract, the roasted Momordica charantia extract has much excellent effect on increasing the expressions of CLOCK, ARNTL, and PER2 genes of hepatic cells, and thus, can further be used for treating or preventing diseases related to CLOCK, ARNTL, and PER2 genes, or regulating physiological functions related to the aforementioned genes, especially be used for adjusting the physiological clock, improving sleep quality, and facilitating sleep.

### BRIEF DESCRIPTION OF REFERENCE NUMERALS

Not applicable.

What is claimed is:

1. A method for increasing the expressions of CLOCK (circadian locomotor output cycles kaput) gene, ARNTL (aryl hydrocarbon receptor nuclear translocator like) gene and/or PER2 (period circadian clock 2) gene in a subject in need, comprising administering to the subject an effective amount of a Momordica charantia extract.

2. The method as claimed in claim 1, which is for treating at least one of cancer, alcohol-induced liver injury, depressive disorder, bipolar disorder, male infertility, aging, Parkinson's disease, vascular disease, metabolic syndrome, and cocaine addiction.

3. The method as claimed in claim 2, wherein the cancer includes gastric cancer, lung cancer, and ovarian cancer.

4. The method as claimed in claim 2, wherein the metabolic syndrome includes obesity, hypertension, and diabetes mellitus.

5. The method as claimed in claim 1, which is for preventing at least one of cancer, alcohol induced liver injury, depressive disorder, bipolar disorder, male infertility, aging, Parkinson's disease, vascular disease, metabolic syndrome, and cocaine addiction.

6. The method as claimed in claim 5, wherein the cancer includes gastric cancer, lung cancer, and ovarian cancer.

7. The method as claimed in claim 5, wherein the metabolic syndrome includes obesity, hypertension, and diabetes mellitus.

8. The method as claimed in claim 1, which is for regulating at least one of glycogen synthesis, lung pathophysiology, maintenance and adaption of skeletal muscles, maintenance and differentiation of pluripotent stem cells, glucose homeostasis, glucose metabolism, insulin release, adipogenesis, retinal function, steroidogenesis, hepatic lipid metabolism, angiogenesis, autophagy in aging, maintenance of bone volume and bone density, circadian clock, diurnal preference, glycogen metabolism, lipid metabolism, endothelial progenitor cell function, and oxidative injury.

9. The method as claimed in claim 1, which is for at least one of adjusting the biological clock, improving sleep quality, and facilitating sleep.

10. The method as claimed in claim 1, wherein the Momordica charantia extract is an extract of roasted Momordica charantia.

11. The method as claimed in claim 10, wherein the roasted Momordica charantia is provided by subjecting Momordica charantia to a roasting treatment at a temperature ranging from 80 to 200° C.

12. The method as claimed in claim 10, wherein the roasted Momordica charantia is provided by subjecting Momordica charantia to a roasting treatment comprising roasting at a stepwise-increased temperature.

13. The method as claimed in claim 12, wherein the roasting treatment comprises a first roasting stage at T1 and a second roasting stage at T2 after the first roasting stage, wherein T1<T2, 80° C.≦T1<200° C., and 80° C.<T2≦200° C.

14. The method as claimed in claim 13, wherein 80° C.≦T1≦135° C., and 135° C.<T2≦200° C.

15. A method for providing a Momordica charantia extract with enhanced effect on increasing the expressions of CLOCK gene, ARNTL gene, and/or PER2 gene, comprising subjecting Momordica charantia to a roasting treatment prior to extracting the Momordica charantia.

16. The method as claimed in claim 15, wherein the roasting treatment comprises roasting the Momordica charantia at a temperature ranging from 80 to 200° C.

17. The method as claimed in claim 15, wherein the roasting treatment comprises roasting the Momordica charantia at a stepwise-increased temperature.

18. The method as claimed in claim 17, the roasting treatment comprises a first roasting stage at T1, and a second roasting stage at T2 after the first roasting stage, wherein T1<T2, 80° C.≦T1<200° C., and 80° C.<T2≦200° C.

19. The method as claimed in claim 18, wherein 80° C.≦T1≦135° C., and 135° C.≦T2≦200° C.
